(19) United States
(12) Patent Application Publication
(10) Pub. No.: US 2020/0353008 A1
(43) Pub. Date: Nov. 12, 2020

(54) COMPOSITIONS AND METHODS FOR TREATING NERVE INJURY

(71) Applicant: MicroVascular Tissues, Inc., San Diego, CA (US)

(72) Inventors: Dale R. PETERSON, CARLSBAD, CA (US); Kevin L. OHASHI, Jamaica Plain, MA (US); Lael J. PICKETT, White Bear Lake, MN (US); Glen GONG, San Carlos, CA (US); Mohammad A. EL-KALAY, Carlsbad, CA (US); Ralph-Heiko MATTERN, Ramona, CA (US)

(21) Appl. No.: 16/960,507

(22) PCT Filed: Jan. 8, 2019
(86) PCT No.: PCT/US2019/012726

§ 371 Date: Jul. 7, 2020

Related U.S. Application Data
(60) Provisional application No. 62/614,948, filed on Jan. 8, 2018.

(51) Int. Cl. A61K 35/28; A61K 35/33; A61K 35/12; A61K 38/18; A61P 25/02; A61K 35/44; A61K 35/35; A61L 27/36; A61K 9/00

(52) U.S. Cl. CPC A61K 35/28; A61K 35/33; A61K 35/12; A61K 38/18; A61K 38/1825; A61K 38/185; A61L 27/3625; A61P 25/02; A61K 35/44; A61K 35/35; A61L 27/3687; A61K 9/0014; A61L 27/3691; A61K 38/1841

(57) ABSTRACT

The disclosure relates to new compositions for the treatment of neuropathy. The compositions of the disclosure provide increased efficacy in the treatment of diabetic neuropathy when applied topically to diabetic ulcers.

### CROSS-REFERENCE TO RELATED APPLICATIONS

The present application claims benefit of priority from U.S. Provisional Patent Application No. 62/614,948, filed on Jan. 8, 2018.

### FIELD

The disclosure relates to compositions and methods for treating nerve injury, including peripheral neuropathy. In particular, the disclosure provides topical and injectable microvascular tissue preparations for treatment of nerve injury.

### BACKGROUND

Nerve injuries are common conditions with broad ranging groups of symptoms depending on the severity and nerves involved. Although much knowledge exists on the mechanisms of injury and regeneration, no reliable treatments that ensure full functional recovery are known.

Among the most common types of nerve injury is so-called peripheral nerve injury, including peripheral neuropathy. Peripheral neuropathy is an all too common condition in which peripheral nerves are damaged or destroyed. The result is a loss of feeling or motor control of a portion of the body or sensations of pain or tingling in the region. If it involves autonomic nerves, then loss of the ability to sweat or to control blood flow in regions of the body may occur. There are many causes including injury, impingement, inflammation, alcohol abuse, medications, and radiation, but by far the largest cause of peripheral neuropathy is diabetes.

One significant cause of peripheral neuropathy is diabetes. It is estimated that there are 389 million diabetics in the world and up to half of them experience diabetic peripheral neuropathy. The loss of sensation often leads to chronic wounds, which are very difficult to heal. If not healed, these chronic wounds open the door to infections, gangrene, and amputations. In the United States, there are 1.2 million diabetic foot ulcers each year leading to 70,000 amputations at a cost of $5.1 Billion.

Trauma is another cause of nerve damage, including peripheral neuropathy. Cut or crushed nerves will occasionally repair spontaneously or with the help of surgical intervention, sometimes using nerve transfers or nerve guide tubes. Another known cause of nerve damage is excessive use of alcohol. Alcohol induced neuropathy can be cured simply by abstaining from alcohol. There is no successful cure for other forms of neuropathy though it is a focus of intense research. The progression of diabetic neuropathy can be slowed by careful control of blood sugar levels, but no one has found a way to reverse the damage. The pain or tingling in many instances of neuropathy can be very hard to live with, and therefore, many treatments aim to relieve these symptoms. Some of the more common treatments include pain relievers, anti-inflammatory drugs, transcutaneous electrical nerve stimulation (TENS), antidepressants, and anti-seizure drugs. In the case of diabetic foot ulcers (DFU), sensation is usually lacking so these treatments are of little value.

Diabetic ulcers are believed to develop primarily because of the lack of sensation caused by the neuropathy. Patients often cannot feel the wounds forming in time to change the loading on the affected area. Once the wound has formed, it is difficult to heal because of the poor blood supply to the tissue, in diabetic patients. Poor blood supply starves the tissue of nutrients needed for healing and allows infections to develop and persist in the wound.

The standard treatment for diabetic ulcers is to debride the wound to remove dead and compromised tissue, use antibiotics to fight infection, cover the wound with hydrocolloid dressings then cover that dressing with an occlusive dressing, and unload the wound. This standard practice leads to healing in only 24% of such wounds. When standard practice fails, there are a few additional FDA approved treatments. The growth factor PDGF-BB has been available for years to improve blood supply to wounds and increase their healing rate. The blood supply can also be improved by repeated hyperbaric oxygen therapy. Both of these established treatments are expensive and still fail to heal many wounds.

A variety of tissue and cell-based therapies have been used to treat DFU. The products and the outcomes vary considerably. The more common tissue products include skin grafts, small intestine submucosa (SIS), and amniotic membranes while the most popular cell therapies are platelet rich plasma (PRP), bone marrow aspirate concentrate (BMAC), adipose stromal vascular fraction (SVF), and mesenchymal stem cells (MSC) cultured from bone marrow or fat. Skin grafts would appear to be the obvious favorite, but they often fail to engraft in ischemic sites and leave wounds at the donor sites. SIS is alleged to provide some useful growth factors as well as wound coverage. Amniotic membranes provide better wound healing results. PRP is prepared by centrifuging some of the patient's blood to enrich platelets 3-7 fold. Similarly BMAC is prepared by drawing bone marrow and centrifuging it to enrich bone marrow stem cells. These two centrifuged products have provided benefits so meager they are hard to measure. In contrast, SVF and MSC provide much larger numbers of multipotent cells and are reported to provide notable wound reversal benefits. None of the many purported existing treatments for DFU provide, however, any noticeable healing of the associated neuropathy.

There remains therefore the need for an effective, affordable treatment for nerve injury, including peripheral neuropathy.

### SUMMARY OF THE INVENTION

The disclosure relates to compositions and methods for treating nerve injury, including peripheral neuropathy. In particular, the disclosure provides topical and injectable microvascular tissue preparations for treatment of nerve injury. In particular, the present inventors have surprisingly discovered that the compositions and methods described herein effectively treat or prevent nerve injury including peripheral neuropathy.

Embodiments of the present invention comprise compositions and methods useful in the repair and/or regeneration of peripheral nerve tissue damage.

Embodiments of the disclosure provide methods of treating or preventing peripheral nerve damage, or promoting tissue regeneration, in a mammal, comprising providing to said mammal a composition of the disclosure. Peripheral nerve damage may be due to injury, impingement, inflammation, alcohol abuse, medications, radiation, or diabetes. In particular embodiments, the peripheral nerve damage is present as a result of a surgical wound, a burn, an injury, a graft wound, or skin lesion. In some embodiments, the peripheral nerve damage is diabetic neuropathy. In further embodiments, the diabetic neuropathy is associated with a diabetic ulcer such as diabetic foot ulcer (DFU).

Embodiments of the disclosure provide compositions comprising therapeutic agents useful in the treatment of peripheral neuropathy. In particular embodiments, the composition is applied topically. In other embodiments, the composition is surgically implanted into the mammal. In certain embodiments, the composition is implanted within or adjacent to a site of injury or disease in said mammal. In related embodiments, the composition is provided to said mammal intravenously.

Therapeutic agents of the disclosure may comprise autograft, allograft, xenograft, microvascular tissue, multipotent cells, microvesicles from such cells or tissues, and/or growth factors. In some embodiments, the composition is sterilized and/or viruses within said composition are inactivated. In particular embodiments, the therapeutic agent is formulated for topical administration and may comprise a powder, film, gel, ointment, suspension, emulsion, or coacervate and the like. In one embodiment, the therapeutic agent is formulated as a dried, hygroscopic powder. In more particular embodiments, the composition is formulated to adhere to a wound. The composition may be applied directly to the wound or at some distance from where the patient has sensation. The composition may be reapplied more than once or at least twice in a three month period. The composition may be reapplied at intervals, for example, of about three (3) to about fifteen (15) days.

In certain embodiments, the microvascular tissue or multipotent cells of the disclosure may be enriched. Compositions of the disclosure may comprise multipotent cells formulated for topical application that provide a dose greater than of about 10⁵ CD90+ cells/cm². In other embodiments, compositions of the disclosure may comprise multipotent cells at a dose of more than about 1000 CFU-F/cm².

In particular embodiments, the composition comprises factors such as growth factors. Growth factors of the disclosure may be transforming growth factor-β1 (TGF-β₁), hepatoma-derived growth factor (HDGF), basic fibroblast growth factor (bFGF), and/or brain-derived neurotrophic factor (BDNF). The growth factors may be in amounts that provide regeneration of peripheral nerves and/or wound healing. Examples of amounts include but are not limited to more than 10 pg of bFGF, BDNF, and/or HDGF and/or less than about 10 pg TGF-β₁ per mg of formulation. One effective composition comprised more than 1 ng/mg of HDGF.

Embodiments of the disclosure provide methods of treating a peripheral nerve damage in a subject in need thereof, comprising: identifying a subject with peripheral nerve damage; and administering a therapeutically effective amount of a therapeutic agent formulated for topical administration. The composition may be applied topically in the affected region or to wounds in the affected region. The methods of treatment of the disclosure may also help heal wounds in addition to the neuropathy.

Embodiments of the disclosure provide methods of treating a diabetic ulcer in a subject in need thereof, comprising: identifying a subject with a diabetic ulcer; and administering a therapeutically effective amount of a composition comprising therapeutic agents of the disclosure formulated for topical administration. Methods of treatment of the disclosure may also heal diabetic ulcers. The diabetic ulcer may be a diabetic foot ulcer. Methods of treatment of the disclosure heal diabetic foot ulcers and reduce the likelihood of a recurrent DFU.

In one aspect, the disclosure provides a method of treating, preventing, improving, or reducing peripheral neuropathy in a subject in need thereof, comprising administering to a subject a composition comprising a therapeutic agent, wherein the subject has at least one damaged nerve in an affected region wherein the administering step comprises topically administering the composition to the affected region.

In another aspect, the disclosure provides composition for treating, preventing, improving, or reducing peripheral neuropathy in a subject in need thereof, comprising a therapeutic agent.

In another aspect, the disclosure provides a method of treating, preventing, improving, or reducing peripheral neuropathy in a subject in need thereof, comprising administering to subject a composition comprising a therapeutic agent, wherein the subject has at least one wound in an affected region, and wherein the administering step comprises topically administering the composition to the wound in the affected region.

### BRIEF DESCRIPTION OF THE DRAWINGS

FIG. 1 shows the lower leg of Patient A, with toes pointed away from the camera, after treatment. Patient A presented with a diabetic foot ulcer on the bottom of his foot that had not healed despite standard care with hydrocolloid dressings. After debridement, the ulcer measured 5.1 cm² and the neuropathy extended halfway to the knee. Four weeks after topical treatment with a multipotent cell preparation (MCP) prepared as described in this disclosure, the DFU measured 3.5 cm² and the extent of the neuropathy was reduced by about 14 cm.

FIG. 2 shows the lower leg of Patient B, with toes pointed towards the camera, after treatment. Patient B presented with a diabetic foot ulcer on the medial side of her foot that had not healed despite standard care with hydrocolloid dressings. After debridement the ulcer measured 6.1 cm² and the neuropathy extended over halfway to the knee. Four weeks after topical treatment with MCP, the DFU measured 0.4 cm² and the extent of the neuropathy was reduced by about 16 cm.

FIG. 3 shows the lower leg of a control subject after treatment with vehicle. Patient C presented with a diabetic foot ulcer on the bottom of her foot that had not healed despite standard care with hydrocolloid dressings. After debridement the ulcer measured 4.0 cm² and the neuropathy extended ⅓ of the distance to the knee. Four weeks after topical treatment with the same vehicle solution used to treat Patients A and B but without MCP, the DFU measured 1.6 cm² but the extent of the neuropathy was not significantly reduced.

### DETAILED DESCRIPTION

The present disclosure relates to compositions and methods useful in the treatment of nerve injury including peripheral nerve damage and peripheral neuropathy, including diabetic neuropathy, and for treatment of diabetic ulcers, including diabetic foot ulcers (DFU).

### Definitions

Unless defined otherwise, all technical and scientific terms used herein have the same meaning as commonly understood by those of ordinary skill in the art to which the invention belongs. For the purposes of the present disclosure, the following terms are defined below.

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

Unless the context requires otherwise, throughout the present specification and claims, the word “comprise” and variations thereof, such as, “comprises” and “comprising” are to be construed in an open, inclusive sense, that is as “including, but not limited to.”

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “one embodiment” or “an embodiment” means that a particular feature, structure or characteristic described in connection with the embodiment is included in at least one embodiment of the disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, the terms “function” and “functional”, and the like, refer to a biological, enzymatic, or therapeutic function.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein.

A “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

By “obtained from” is meant that a sample such as, for example, a cell or tissue, is isolated from, or derived from, a particular source, such as a desired organism or a specific tissue within a desired organism.

As used herein, unless the context makes clear otherwise, “treatment,” and similar words such as “treated,” “treating” etc., indicates an approach for obtaining beneficial or desired results, including clinical results. Treatment can involve optionally either the reduction or amelioration of symptoms of an injury, disease or condition, or the delaying of the progression of the injury, disease or condition. Administration of a composition described herein may, in some embodiments, treat one or more of such symptoms.

As used herein, unless the context makes clear otherwise, “prevention,” and similar words such as “prevented,” “preventing” etc., indicates an approach for preventing, inhibiting or reducing the likelihood of the onset or recurrence of an injury, disease or condition. It also refers to preventing, inhibiting or reducing the likelihood of the occurrence or recurrence of one or more symptoms of an injury, disease or condition, or optionally an approach for delaying the onset or recurrence of an injury, disease or condition or delaying the occurrence or recurrence of one or more symptoms of an injury disease or condition. As used herein, “prevention” and similar words also includes reducing the intensity, effect, symptoms and/or burden of an injury, disease or condition.

As used herein, an “effective amount” or a “therapeutically effective amount” of a composition is that amount sufficient to affect a desired biological effect, such as, e.g., beneficial clinical results.

As used herein, “diabetes” is a metabolic disease in which the body's cells are unable to properly metabolize glucose. In Type I diabetes islet cells are destroyed in the pancreas so that insulin levels in the body are inadequate for glucose metabolism. In Type II diabetes the patient's cells become refractory to insulin and so lose their ability to properly use glucose.

As used herein, “nerve injury” refers to damage, disease, malfunction or injury to nerves from any cause, including, but not limited to, diabetes, alcohol, ischemia, and trauma. Types of trauma include blunt-force trauma, puncture wounds, and lacerations. Ischemia may be caused by inadequate blood supply or other factors. Nerve injury therefore refers generally to any intentional or unintentional, disease-related or not disease-related, localized or systemic, surface or deep-tissue injury that causes the nerves of a subject to fail to function to the same extent as nerves function in a healthy subject. As used herein, “neuropathy” refers to specific forms of nerve injury, such as caused by nerve damage or disease. Neuropathy may be evidenced by effects on sensation (pain, tingling, prickling, numbness), motion (muscle weakness or wasting), or organ function (sweat glands, blood vessel tone) of the subject. The term “peripheral neuropathy” refers to forms of neuropathy that effect peripheral nerves. Peripheral neuropathy may include either diabetic or non-diabetic neuropathy.

As used herein, “affected region” refers to the area of the subject's body that experiences aberrant sensation or response to stimuli due to nerve damage. An affected region may be a region within about 5, 10, 15, 20, 25, or 30 cm of a damaged nerve. The dimensions of the affected region are determined by the type or severity of damage to the damaged nerve.

As used herein, “diabetic neuropathy” refers to the most common form of peripheral neuropathy. It is due to diabetes and usually effects the distal limbs. Without being bound by theory, diabetic neuropathy is thought to originate, in many cases, because of reduced blood flow to the nerves. It is marked by gradual numbness, tingling, or pain and may move up the patient's legs and arms.

As used herein, the term “wound” refers to a break through the skin. It may be an acute breach of the skin or a chronic defect in the barrier provided by the skin (also referred to as an ulcer). Wounds include puncture wounds, laceration, abrasions, and skin degradation by other mechanisms. Wounds include intentional (e.g. surgical) wounds and unintentional injury, such as caused by disease or by accidental injury.

As used herein, the term “isolated,” e.g., with respect to a multipotent cell, means removed from its natural environment. For example, a cell is isolated if it is separated from some or all of the coexisting materials in its natural environment.

The term “processed microvascular tissue” as used herein refers to microvascular tissue that is dissociated into small fragments. In some embodiments, processed microvascular tissue is dried, irradiated, or formulated. In some embodiments, processed microvascular tissue comprises multipotent cells.

As used herein, “multipotent cells” refers to cells that maintain the capacity to differentiate into two or more different specialized cell types. Multipotent cells include stem cells and multipotent progenitor cells. Examples of multipotent cells include, but are not limited to, mesenchymal stem cells, embryonic stem cells, neural stem cells, endothelial progenitor cells, adipose-derived stem cells, vascular pericytes and umbilical cord stem cells. It is understood that following sterilization or preservation according to the methods described herein, a multipotent cell may lose its capacity to grow, proliferate, or differentiate. The terms “multipotent cell preparation” and “MCP” refer to multipotent cells that are isolated from at least some of the coexisting material found in the natural state and processed enzymatically, chemically, physically, or by irradiation to alter the cells contained therein from their state in the source tissue or to increase their concentration.

As used herein “mVASC®” refers to a multipotent cell preparation product supplied by Microvascular Tissues, Inc. mVASC® is sterile, off-the-shelf human allograft microvascular tissue.

The terms “autologous transfer,” “autologous transplantation,” and the like refer to treatments wherein the tissue donor is also the recipient of the composition produced from the tissue.

The terms “allogeneic transfer,” “allogeneic transplantation,” and the like refer to treatments wherein the tissue donor is of the same species as the recipient of the composition produced from the tissue, but is not the same individual.

The terms “xenogeneic transfer,” “xenogeneic transplantation,” and the like refer to treatments wherein the tissue donor is of a different species than the recipient of the composition produced from the tissue.

As used herein, the term “enriched” refers to a process of increasing the concentration or the frequency in a mixture of a target such as multipotent cells. This could be accomplished by removing extraneous cells, tissue or fluid from the multipotent cells and/or by expanding them in cell culture.

As used herein, the term “therapeutic agent” refers to a composition intended for the treatment of nerve injury, e.g., peripheral neuropathy, that may include drugs, biologics, tissues, and/or cells. In some embodiments, the therapeutic agent is an MCP or an MCP in combination with one or more drugs, biologics, tissues, and/or cells. The composition may also contain stabilizers, emulsifiers, adhesives and other pharmaceutical formulants.

As used herein, the term “growth factors” refers to a naturally occurring substance capable of stimulating cellular growth, proliferation, healing, and cellular differentiation. Usually it is a protein or a steroid hormone. Growth factors are important for regulating a variety of cellular processes. Growth factors typically act as signaling molecules between cells. Examples are cytokines and hormones that bind to specific receptors on the surface of their target cells. They often promote cell differentiation and maturation, which varies between growth factors.

As used herein, “brain-derived neurotrophic factor”, or “BDNF”, is a member of the neurotrophin family of growth factors, which are related to the canonical Nerve Growth Factor. Neurotrophic factors are found in the brain and the periphery.

As used herein “basic fibroblast growth factor” or “bFGF”, also known as FGF2 or FGF-β, is a member of the fibroblast growth factor family. bFGF mediates angiogenesis and is a critical component of human embryonic stem cell culture medium; the growth factor is necessary for the cells to remain in an undifferentiated state.

As used herein, “hepatoma-derived growth factor” or “HGDF” refers to a protein that exhibits proliferative, angiogenic, and neurotrophic activity and is suggested to be involved in organ development. It is also known as high mobility group protein 1-like 2 (HMG-IL2).

As used herein, “transforming growth factor beta 1” or “TGF-β1” is a polypeptide member of the transforming growth factor beta superfamily of cytokines. It is a secreted protein that performs many cellular functions, including the control of cell growth, cell proliferation, cell differentiation and apoptosis.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent or emulsifier which has been approved by the United States Food and Drug Administration as being acceptable for use in humans or domestic animals.

A “pharmaceutical composition” refers to a formulation of a composition of the invention and a medium generally accepted in the art for the delivery of a therapeutic agent to mammals, e.g., humans. Such a medium includes any pharmaceutically acceptable carriers, diluents or excipients therefore.

In one aspect, the disclosure provides a method of treating, preventing, improving, or reducing peripheral neuropathy in a subject in need thereof, comprising administering to a subject a composition comprising a therapeutic agent, wherein the subject has at least one damaged nerve in an affected region wherein the administering step comprises topically administering the composition to the affected region.

In another aspect, the disclosure provides composition for treating, preventing, improving, or reducing peripheral neuropathy in a subject in need thereof, comprising a therapeutic agent.

In another aspect, the disclosure provides a method of treating, preventing, improving, or reducing peripheral neuropathy in a subject in need thereof, comprising administering to subject a composition comprising a therapeutic agent, wherein the subject has at least one wound in an affected region, and wherein the administering step comprises topically administering the composition to the wound in the affected region.

In some embodiments, the therapeutic agent comprises, per milliliter, one or more cells or proteins selected from at least about 1000 CFU-F cells, the contents of at least about 1000 CFU-F cells, at least about 10⁵ CD90 positive cells, the contents of at least about 10⁵ CD90 positive cells, at least about 10 picograms (pg) HDGF, at least about 10 pg bFGF, at least about 10 pg BDNF, at least about 10 pg of bFGF and BDNF, collectively, and at most about 10 pg TGF-b1.

In some embodiments, the subject suffers from diabetes or the peripheral neuropathy comprises diabetic neuropathy.

In some embodiments, the subject has one or more diabetic foot ulcers (DFU).

In some embodiments, the administering step comprises topically administering the composition to the subject at least twice in three months.

In some embodiments, the administering step comprises topically administering the composition to the subject every 3, 4, 5, 6, 7, 8, 9, 10, 11, 12, 13, 14, 15, 16, 17, 18, 19, or 20 days.

In some embodiments, the affected region comprises a wound, optionally an ulcer.

In some embodiments, the therapeutic agent comprises a multipotent cell preparation (MCP) selected from the group consisting of processed microvascular tissue, a mesenchymal stem cell (MSC) preparation, stromal vascular fraction (SVF) from adipose tissue, a bone marrow cell preparation, and umbilical cord stem cells.

In some embodiments, the therapeutic agent comprises processed microvascular tissue, optionally mVASC®.

In some embodiments, the processed microvascular tissue is derived from human adipose tissue.

In some embodiments, the processed microvascular tissue is processed with one or more treatment selected from collagenase treatment, neutral protease treatment, cell lysis, optionally ammonium chloride cell lysis, drying, optionally freeze-drying, air drying, or lyophilization, freezing, optionally freeze-drying or flash freezing, and irradiation, optionally gamma irradiation or electron-beam irradiation.

In some embodiments, the composition is formulated for topical administration.

In some embodiments, the composition comprises a powder, a crushable cake, a film, a gel, an ointment, a suspension, an emulsion, a concentrate, a coacervate, a scaffold, a hygroscopic powder, or a wound dressing or covering.

In some embodiments, administering the composition to the subject results in adherence of the composition to the affected region.

In some embodiments, the method results in one or more of improved nerve sensation, tissue healing, wound closure, ulcer healing, prevention of wound recurrence, increased flexibility, or decreased pain.

In some embodiments, the method improves healing of the ulcer, improves nerve sensation in the region, or reduces the recurrence rate of new ulcers in the region.

In some embodiments, the affected region is within 5, 10, 15, 30 cm of a damaged nerve.

Therapeutic Agent Compositions

The present disclosure provides therapeutic agents for treating and/or preventing nerve injury, such as neuropathy.

There are many potential sources of multipotent cells. Table 1 lists many of the common sources along with the frequency of the desired cells in the source tissues.

TABLE 1
Frequency of Stem Cells in Various Tissues

| | | Nucleated | | # Marker | |
|---|---|---|---|---|---|
| | | Cells/gm | | Positive | CFU- |
| Source | Description | Source | Marker | Cells/gm | F/gram |
| Blood | Adult | NR | CD34 | 110 | NR |
| | Adult | ~1 × 10⁶ | NR | NR | 1 |
| | Fetal | ~1 × 10⁶ | | NR | 8 to 9 |
| Amniotic | 10 wk | 4000 | CD117 | 36 | 4 |
| Fluid | Full term | 7,300 | CD117 | 22 | 1 |
| Amnion | 48 wk, Full | 400,000 | TRA1- | 4,000 | NR |
| | term | | 60 | | |
| Placenta | Full Term, 15 | NR | NR | NR | 20-33 |
| | g samples | | | | |
| Umbilical | 36 wk, full | ~1 × 10⁶ | NR | NR | 800 |
| Cord | term | | | | |
| Bone | 29 donors | 8,600,000 | NR | NR | 666 |
| Marrow | 40 donors | 16,600,000 | NR | NR | 898 |
| | 6 donors | 55,000,000 | NR | NR | 1980 |
| | 6 donors | 27,000,000 | CD34 | 5,500 | NR |
| Adipose | 66 donors | 350,000 | NR | NR | 7,000 |
| | 8 donors | NR | CD34 | 300,000 | NR |
| | 18 donors | 400,000 | NR | NR | NR |
| | 5 donors/4 | 100,000 to | NR | NR | 4,000 |
| | instruments | 500,000 | | | |

The multipotent cells are frequently used along with the rest of their native tissue, sometimes they are isolated from the tissues, and many times they are cultured to expand their numbers. Methods of preparation of multipotent cells from various tissues are known in the art. Typically, protocols for growing mesenchymal stem cells (MSC) are used to expand the number of multipotent cells.

Multipotent cell products, Platelet Rich Plasma (PRP) or Bone Marrow Aspirate Concentrate (BMAC) have been used to treat DFU by injection into the wound bed or around the periphery of the wound or applied with fibrin glue to hold the cells at the site. Such treatments have been reported to improve wound healing. Such application of PRP and BMAC has shown no effect on neuropathy.

Multipotent cells have been injected into the tissue adjacent to damaged nerves or provided systemically as treatment for neuropathy. When nerve damage is treated surgically, multipotent cells have been included in the nerve guide tubes used to help repair the nerve. There are some reports of success with such treatments, but not in diabetic neuropathy patients.

Freeze-dried and sterilized multipotent cells maintain or even enhance therapeutic benefits. Relevant methods for preparation these types of multipotent cells are provided in U.S. patent application Ser. No. 15/633,311; U.S. patent application Ser. No. 14/429,511; and International Patent Appl. No. PCT/US2013/060181. In some cases, such multipotent cells are 1) stable for years when stored at room temperature, 2) ready to use in a minute or less, 3) effective in allogeneic or even xenogeneic recipients, 4) present no risk of disease transmission, and 5) present no danger of inappropriate differentiation or expansion of the cells.

The present disclosure presents surprising results with multipotent cell preparations. The present inventors have demonstrated unexpected success with topical application of multipotent cells for the treatment of nerve injury. In some embodiments, the disclosure provides methods related to applying large doses of multipotent cells topically to regions affected by damaged nerves (e.g., diabetic wounds). The present inventors observed a distinct improvement in healing of the DFU and surprisingly a substantial reversal of the peripheral neuropathy in the treated limb.

Various methods for determining the dose of multipotent cells are provided by the present disclosure. In some embodiments, the multipotent cells of the disclosure comprise large doses of cells that are CD34+, CD90+, CD117+, or CD271+. In some embodiments, the multipotent cells of the disclosure comprise large doses of double, triple, or quadruple positive cells using the markers CD34, CD90, CD117, CD271, or other markers for stem cells known in the art. The present inventors have observed that in some cases, quantitating cells using these methods overestimates the number of stem cells. Thus, in some embodiments, the method of the disclosure comprises quantifying stem cells in a multipotent cell preparation by in a limiting dilution assay by counting the number of fibroblast-like colonies (CFU-F) formed. The present inventors observe, however that this CFU-F method in some cases underestimates the number of stem cells because some may not grow in the culture conditions. Thus, in some embodiments, the method comprises assaying the multipotent cell preparation for CFU-F and for CD90+ cells. In some embodiments, the compositions of the disclosure comprise at least 1000 CFU-F or 10⁵ CD90+ cells per square cm of wound treated.

When we applied these processed multipotent cells to DFU at more than 1000 CFU-F or 10⁵ CD90+ cells per square cm of wound treated, dramatic improvement in wound healing and a reversal of the peripheral neuropathy in that treated limb was observed. The greatest improvement in healing and in neuropathy was seen when the processed multipotent cells were applied topically directly to the DFU. In other cases, there is improvement when processed multipotent cells are injected locally.

In some embodiments, the therapeutic agent comprises one or more of processed microvascular tissue, multipotent cells, a multipotent cell preparation (MCP) and other components, such as without limitation, cytokines and growth factors. In some embodiments, the compositions of the disclosure comprise multipotent cells having intact cell membranes (“intact multipotent cells”). In some embodiments, the compositions of the disclosure do not comprise intact multipotent cells. In some embodiments, the compositions of the disclosure comprise multipotent cells, wherein at most about 10%, about 20%, about 30%, about 40%, about 50%, about 60%, or about 70% of the multipotent cells have intact cell membranes. In some embodiments, the compositions of the disclosure comprise multipotent cells, wherein at most about 10%, about 20%, about 30%, about 40%, about 50%, about 60%, or about 70% of the multipotent cells are viable, that is “live multipotent cells.” The compositions of the disclosure may or may not comprise intact multipotent cells. In particular embodiments, a composition of the disclosure does not comprise any intact multipotent cells or does not comprise any live multipotent cells or does not comprise any live cells. In certain embodiments, a composition of the disclosure comprises fragments or cell membranes of multipotent cells. A composition of the disclosure may comprise live and/or dead multipotent cells.

Compositions of the disclosure may be prepared from any mammalian tissue, e.g., tissue obtained from a mammal, such as a human, a non-human primate, a dog, a cat, or a horse. The compositions of the disclosure may be used to treat an autologous, allogeneic or xenogeneic subject. Accordingly, agents, cells, or tissue may be obtained from the subject to be treated, or from a different donor subject (e.g. a human or other animal), which may be the same or a different species as the subject to be treated (e.g., where the subject is human, the donor may be anon-human animal, e.g. a primate). In particular embodiments, the agent, cells, or tissue is obtained from an allogeneic donor of the same species as the subject to be treated, e.g., a human or non-human mammalian donor. In particular embodiments, the donor animal is a healthy donor. In some embodiments, the composition comprises one or more growth factors at concentrations greater than that observed in the source tissue. In some embodiments, the growth factor is derived from the same source tissue as the other components of the composition but enriched by processing (i.e. the growth factors are endogenous to the source tissue). In some embodiments, the growth factor is exogenous—i.e. derived from a source other than the source issue. Growth factors may be obtained from commercial sources, may be made recombinantly (e.g. in bacterial, insect-cell, or mammalian expression systems), or may be isolated from natural source (e.g. a donor). Growth factors or other biologics in the composition may be isolated from tissues or harvested from cells or microbes grown in culture.

In various embodiments, microvascular tissue compositions or multipotent cell are prepared from any of a number of different tissues. In particular embodiments, the tissue is non-embryonic tissue. For example, in particular embodiments, the tissue used to prepare the compositions of the disclosure is a vascular tissue or a microvascular tissue, such as, e.g., adipose tissue, skin, bone, tendon tissue, post-partum tissue, bone marrow, or muscle tissue.

In certain embodiments, the composition of the disclosure comprises one or more multipotent cells, differentiated cells, components of the extracellular matrix, growth factors, angiogenic agents, anti-inflammatory agents, cytokines, chemokines, growth factors, and/or differentiation agents. Extracellular matrix components include but are not limited to extracellular matrix proteins, such as various collagens, fibronectin, vitronectin, and thrombospondin, and others described herein.

In various embodiments, the shelf life of the composition is at least about one week, at least about one month, at least about two months, at least about six months, or greater at room temperature while maintaining one or more biological activities. In particular embodiments, the composition retains measurable angiogenic, anti-inflammatory, and/or tissue healing activity when stored at approximately 4° C. for at least about one month, at least about two months, at least about four months, at least about six months, or at least about one year. In particular embodiments of the compositions described herein, the composition retains measurable angiogenic, anti-inflammatory, or tissue healing activity when stored at approximately −20° C. for at least about one month, at least about two months, at least about four months, at least about six months, or at least about one year. In particular embodiments, the measurable angiogenic, anti-inflammatory, or tissue healing activity is at least about 10%, at least about 20%, at least about 30%, at least about 40%, at least about 50%, at least about 60%, at least about 70%, at least about 80%, or at least about 90% of the activity prior to storage, when measured in an in vivo or in vitro assay, including any of those described herein.

Dissociated tissue, or cells and other tissue components isolated therefrom, including the resulting compositions, are optionally sterilized, e.g., to reduce or eliminate contamination by microorganisms, such as, e.g., bacterial, viruses, and fungi, or prions. In particular embodiments, compositions comprising multipotent cells and/or other tissue components, are sterilized using irradiation. Methods of sterilization exist using radiation such as electron beams, X-rays, gamma rays, or ultraviolet radiation. In particular embodiments, sterilization is performed by exposing dissociated tissue, or cells and other tissue components isolated therefrom, to gamma radiation at a dosage in the range of about 0.5 to about 5.0 Mrad, or about 1.0 to about 3.0 Mrad, or about 1.0 Mrad, or about 1.5 Mrad, or about 2.0 Mrad, or about 2.5 Mrad, or about 3.0 Mrad, or about 3.5 Mrad, or about 4.0 Mrad, or about 4.5 Mrad, or about 5.0 Mrad (or any amount of gamma radiation between those values). In particular embodiments, sterilization is performed by exposing dissociated tissue, or cells and other tissue components isolated therefrom, to electron beam radiation at a dosage in the range of about 0.5 to about 5.0 Mrad, or about 1.0 to about 3.0 Mrad, or about 1.0 Mrad, or about 1.5 Mrad, or about 2.0 Mrad, or about 2.5 Mrad, or about 3.0 Mrad, or about 3.5 Mrad, or about 4.0 Mrad, or about 4.5 Mrad, or about 5.0 Mrad (or any amount of electron beam radiation between those values). It is often easier to measure the amount of radiation to which the compositions are exposed than the amount of radiation generated by the source. In particular embodiments, E-beam or gamma radiation levels for sterilization are about 9 kGy to about 50 kGy, or about 9 kGy to about 20 kGy, or about 20 k to about 30 kGy, or about 30 kGy to about 40 kGy (or any amount of radiation between those values). In some embodiments, E-beam or gamma radiation levels for sterilization are about 9 kGy to about 17 kGy. In some embodiments, the composition comprises MCP processed by sterilizing irradiation. In some embodiments, the composition comprises processed microvascular tissue processed by sterilizing irradiation.

In addition, dissociated tissue, or cells and other tissue components isolated therefrom, may be treated to inactivate viruses. Methods of inactivating viruses are known in the art, including the use of irradiation, as described above for sterilization. Other methods of inactivating viruses may be used, including acid or base treatments, bleach, aldehyde or ethylene oxide solutions, or heat. It is understood that cryprotectants and other excipients used for lyophilizing or freezing the composition may also protect against radiation. For example, sugars and albumin (or other stabilizing proteins) along with the low temperature protect against radiation damage to cells. Accordingly, in particular embodiments, sterilization or viral inactivation is performed after lyophilization.

In particular embodiments, less than about 50%, less than about 40%, less than about 30%, less than about 20%, less than about 10%, or less than about 5% of the cells present in a composition of the disclosure are viable. In several embodiments, substantially all of the cells are non-viable. As used herein, the term “viable” shall be given its ordinary meaning and shall also refer to a cell that is capable of proliferating when cultured under appropriate conditions, e.g., conditions under which the same cell or type of cell would be expected to proliferate, e.g., if not processed as described herein. In other embodiments, less than about 2% or less than about 1% of the cells present in said composition are viable. In particular embodiments, none or substantially none of the cells present in the composition are viable. Accordingly, the term “non-viable’ means that the cell is not capable of proliferating when cultured under appropriate conditions, e.g., conditions under which the same cell would be expected to proliferate, e.g., if not processed as described herein.

It is understood according to the disclosure that, although cells within the compositions described herein may not be viable and may not persist long after being transplanted into a subject, the compositions trigger a cascade of responses in the subject that lead to improved healing, reduced inflammation, or increased angiogenesis. The multipotent cell preparation (MCP) and processed microvascular tissue compositions described in this disclosure need not include viable or whole stem cells to promote or induce healing of injured or diseased tissue, such as, e.g., nerve inury, such as peripheral nerve tissue. In addition, the compositions of the disclosure may comprise processed tissue and various components thereof, including dissociated tissue, cells, such as multipotent cells (e.g., stem cells), cell membranes, extracellular matrix components, and various growth factors, angiogenic factors, anti-inflammatory agents, cytokines, differentiation agents, etc. present within or associated with a tissue sample used to prepare the compositions. The composition may also be prepared from components obtained from other sources.

In some embodiments, compositions administered by the methods of the disclosure may comprise live multipotent cells, drugs, and/or biological factors such as a growth factor, an angiogenic agent, an anti-inflammatory agent, a cytokine, or a differentiation agent. For example, a growth factor or angiogenic agent may be selected from basic fibroblast growth factor, other fibroblast growth factors, bone morphogenetic proteins, hepatocyte growth factor, keratinocyte growth factor, granulocyte macrophage colony stimulating factor, platelet-derived growth factor, transforming growth factor β1 and/or β3, or vascular endothelial cell growth factor. Additional therapeutic agents found endogenously or added exogenously to the composition of the disclosure include any of those listed in Table 2. In one embodiment of the disclosure, the therapeutic agent composition comprises bFGF, BDNF, TGF-β1, HDGF, or a combination thereof.

TABLE 2
Illustrative and Non-Limiting List
of Additional Therapeutic Agents

| Factor | Activity |
|---|---|
| IL-1a | Inflammatory cytokine |
| IL-1ra | Inflammatory inhibitor |
| HGF | Angiogenesis |
| HDGF | Angiogenesis, neurogenesis |
| MMP-1 | Matrix metalloproteinase |
| MMP-3 | Matrix metalloproteinase |
| BDNF | Neurogenesis |
| GDNF | Neurogenesis |
| CNTF | Neurogenesis |
| G-CSF | Hematopoiesis, inflammation |
| GM-CSF | Hematopoiesis, inflammation |
| OPG | |
| PLGF | Angiogenesis |
| ANG | Angiogenesis |
| ANGPT-2 | Angiogenesis |
| MCP-1 | Cell migration |
| SDF-1α | Cell migration |
| PGE₂ | |
| ICAM | Cell migration |
| VCAM | Cell migration |
| PECAM | Cell migration |
| c-myc | |
| EGF | Cell proliferation |
| TNF- α | inflammation |
| VEGF | Angiogenesis, neurogenesis |
| PDGF | Cell proliferation, angiogenesis |
| FGF-1 | Cell proliferation |
| BMP-2 | Bone formation |
| BMP-4 | Bone formation |
| BMP-13 | Bone and cartilage |
| IL-4 | Inflammation |
| IL-6 | Inflammation |
| IL-8 | Inflammation |
| IL-10 | Inflammation |
| NGF | Neurogenesis |
| MIP-1α | Cell migration |
| MIP-1β | Cell migration |
| OPN | Bone formation |

In some embodiments, the therapeutic agent is selected from the group consisting of nerve growth factor (NGF), glial cell line derived neurotrophic factor (GDNF), J147, curcumin, and Insulin-like growth factor 1 (IGF-1) or a derivative thereof.

As used herein “NGF” refers a neurotrophic factor and neuropeptide primarily involved in the regulation of growth, maintenance, proliferation, and survival of certain target neurons. NGF is initially expressed in a 130-kDa complex of three proteins (α-NGF, β-NGF, and γ-NGF) termed proNGF. The gamma subunit of this complex acts as a serine protease, cleaving the N-terminal of the beta subunit, thereby activating the protein into functional NGF. In embodiments, the therapeutic agent is either proNGF or NGF.

As used herein “GDNF” refers to a protein that, in humans, is encoded by the GDNF gene. GDNF is a small protein that potently promotes the survival of many types of neurons. It signals through GFRα receptors, particularly GFRα1.

J147 is an experimental drug with reported effects against both Alzheimer's disease and ageing in mouse models of accelerated aging. J147 has the following chemical structure:

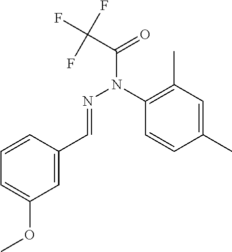 [SMILES: COc1cccc(/C=N/N(C(=O)C(F)(F)F)c2ccc(C)cc2C)c1]

Curcumin is a bright yellow chemical produced by some plants. It is the principal curcuminoid of turmeric. Curcumin has the following chemical structure:

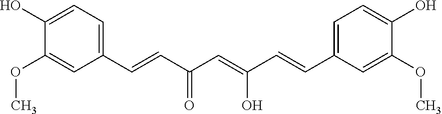 [SMILES: COc1cc(/C=C/C(=O)/C=C(O)/C=C/c2ccc(O)c(OC)c2)ccc1O]

Curcumin and its derivatives have been suggested as treatments for neurodegenerative diseases including Alzheimer's disease (AD), and Parkinson's disease (PD) and malignancy. (Lee et al. Curr Neuropharmacol. 2013 July; 11(4): 338-378).

As used herein “IGF-1” refers a protein that in humans is encoded by the IGF1 gene. IGF-1 is a hormone similar in molecular structure to insulin. IGF-1 binds to at least two cell surface receptor tyrosine kinases: the IGF-1 receptor (IGF1R) and the insulin receptor. IGF-1 is one of the most potent natural activators of the AKT signaling pathway, a stimulator of cell growth and proliferation, and a potent inhibitor of programmed cell death.

Compositions of the disclosure, in some embodiments, promote healing of an injured or diseased tissue. In some embodiments, the compositions of the disclosure have tissue healing activity. As used herein, “tissue healing activity” of a composition is the ability of the composition to facilitate improved healing (e.g., repair or regeneration) of an injured or diseased tissue (e.g., peripheral nerve damage) exposed to the composition as compared to an analogous tissue similarly treated but without exposure to the composition, or to a control composition (or “vehicle”) comprising the same or similar components as the test composition without one or more therapeutic agents (such as the MCP or processed microvascular tissue). Improved healing is measured using any appropriate means, including but not limited to time to complete healing, amount of new tissue generated, strength of the resulting healed tissue, or functionality of the resulting healed tissue. Examples of appropriate measures include but are not limited to gain or improvements of limb sensation or wound repair of a diabetic ulcer. In particular embodiments, a composition of the disclosure has one or more biological activities. For example, in certain embodiments, a composition has anti-inflammatory or angiogenic activity. In certain related embodiments, a composition promotes blood vessel formation or tissue healing. Combinations of these effects are achieved in several embodiments.

In certain embodiments, a composition of the disclosure has anti-inflammatory activity. In particular embodiments, an injured or diseased tissue (e.g., an injured or diseased tissue undergoing an inflammatory response) exposed to or contacted with a composition of the disclosure exhibits reduced inflammation as compared to when the injured or diseased tissue is similarly treated but not exposed to or contacted with the composition of the disclosure. In certain embodiments, the amount of inflammation in the tissue exposed to or contacted with the composition of the disclosure is reduced by at least about 10%, at least about 20%, at least about 30%, at least about 40%, at least about 50%, at least about 60%, at least about 70%, at least about 80%, or at least about 90%, as compared to the amount of inflammation when the injured or diseased tissue is not exposed to or contacted with the composition of the disclosure. Inflammation may be measured by any means available in the art, including but not limited to, e.g., the number of lymphocytes observed in the affected tissue when observed histologically.

In particular embodiments, a composition of the disclosure has anti-inflammatory activity that may be measured in an in vitro assay. In certain embodiments, the amount of inflammation measured in an in vitro assay in the presence of a composition of the disclosure is at least about 10%, at least about 20%, at least about 30%, at least about 40%, at least about 50%, at least about 60%, at least about 70%, at least about 80%, or at least about 90% less than the amount of inflammation measured in the same assay in the absence the composition of the disclosure or in the presence of a control composition. In particular embodiments, the in vitro assay is a mixed lymphocyte reaction.

In certain embodiments, a composition of the disclosure has angiogenic activity. In particular embodiments, an injured or diseased tissue (e.g., an injured or diseased tissue undergoing an inflammatory response) exposed to or contacted with a composition of the disclosure exhibits increased angiogenesis as compared to when the injured or diseased tissue is similarly treated but not exposed to or contacted with the composition of the disclosure. In certain embodiments, the amount of angiogenesis in the tissue exposed to or contacted with the composition of the disclosure is increased by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, or at least about 500%, as compared to the amount of angiogenesis when the injured or diseased tissue is not exposed to or contacted with the composition of the disclosure. Angiogenesis may be measured by any means available in the art, including but not limited to, e.g., the hindlimb ischemia model.

In particular embodiments, a composition of the disclosure has angiogenic activity that may be measured in an in vivo or in vitro assay. In certain embodiments, the amount of activity measured in an in vitro angiogenesis assay in the presence of a composition of the disclosure is at least about 10%, at least about 20%, at least about 30%, at least about 40%, at least about 50%, at least about 60%, at least about 70%, at least about 80%, at least 90 about %, at least about 100%, at least about 150%, at least about 200%, at least about 300%, at least about 400%, or at least about 500% greater than the amount of activity measured in the same assay in the absence the composition of the disclosure or in the presence of a control composition. In particular embodiments, the in vivo assay is a matrigel implant assay. In particular embodiments, the in vitro assay is an endothelial cell migration assay. In some embodiments, the in vitro assay is a tube formation assay, an invasion assay, or a node assay.

For the purposes of administering a composition of the invention to a subject in need thereof, the compositions may be formulated as pharmaceutical compositions. Pharmaceutical compositions of the disclosure comprise a composition of the disclosure and a pharmaceutically acceptable excipient, carrier and/or diluent. The composition of the invention is present in the pharmaceutical composition in an amount sufficient to effect treatment or prevention of an injury, disease or disorder in a subject in need thereof, i.e., in a therapeutically effective amount.

Pharmaceutically acceptable excipients, carriers and/or diluents are familiar to those skilled in the art. For compositions formulated as liquid solutions, acceptable carriers and/or diluents include saline and sterile water, and may optionally include antioxidants, buffers, bacteriostats and other common additives. The pharmaceutical compositions of the invention can be prepared by combining a composition of the invention with an appropriate pharmaceutically acceptable carrier, diluent or excipient, and may be formulated into preparations in solid, semi-solid, liquid or aerosol forms, such as powders, granules, solutions, injections, inhalants, and microspheres. These compositions may also contain dispersing and surface active agents, binders and lubricants. One skilled in this art may further formulate a composition of the invention in an appropriate manner, and in accordance with accepted practices, such as those disclosed in Remington's Pharmaceutical Sciences, Gennaro, Ed., Mack Publishing Co., Easton, Pa. 1990. In some embodiments, the composition is formulated for topical administration, for example as a powder, cream, ointment, or other topical composition.

Methods of Use

The disclosure further provides methods of treating or preventing nerve injury, such as neuropathy using any of the compositions or therapeutic agents of the disclosure. In some embodiments, the methods comprise administering a composition comprising one or more therapeutic agents to a subject suffering from or suspected of suffering from nerve damage. In some embodiments, the nerve injury is peripheral neuropathy. In some embodiments, the nerve injury is diabetic neuropathy. In some embodiments, the administering step comprises injecting the composition into the subject at or near the site of nerve injury. In some embodiments, the administering step comprises applying the composition topically on or in the area of the nerve injury, peripheral nerve damage, or peripheral neuropathy. In some embodiments, the composition is applied topically in the area of neuropathy or to a wound in the region affected by the neuropathy. In some embodiments, the subject suffers from DFU and the composition is injected into the foot. In some embodiments, the subject suffers from DFU and the composition is topically applied to the foot. In some embodiments, the subject suffers from DFU, the composition comprises MCP and, optionally, one or more other therapeutic agents, and the composition is topically applied to the ulcer on the foot.

Routes of administering the compositions of the disclosure include, without limitation, topically, intramuscular, intravenous, intraarterial, intraperitoneal, subcutaneous, oral, nasal, transplantation, implantation, injection, delivery via a catheter, topical, transdermal, inhalation, parenteral, and intranasal. The composition may be administered in a matrix, gel, or other scaffold. The term parenteral as used herein includes subcutaneous injections, intravenous, intramuscular, intrasternal injection or infusion techniques. In addition, the compositions of the invention may be surgically implanted, injected, delivered (e.g., by way of a catheter or syringe), or otherwise administered directly or indirectly to the site in need of repair or augmentation. For example, compositions of the disclosure may be surgically introduced into or adjacent to a site of injury or disease in a subject. In some embodiments, administration is intravenous. The composition may be formulated for a particular route of administration. In particular embodiments, the method is surgically for tissue repair, intravenously for treatment of ischemia, injection into joint spaces for treatment of pain and inflammation, topically to wounds, and injection into muscle for treatment of peripheral vascular disease.

The present disclosure contemplates topical administration to a wound, such as a diabetic foot ulcer. In some embodiments, topical administration results in improved treatment and/or wound healing compared to injection of the same or similar composition into the same or similar wound. In some embodiments, topical administration to a DFU results in better treatment of the DFU than injection of the same or similar composition into the foot. However, although compositions of the invention may be administered by many routes, another important aspect of this invention is the discovery that topical delivery of therapeutic agents to a wound in the area affected by neuropathy was far more effective at treating the neuropathy than other routes of administration. Topical administration may be performed close to or far from healthy nerves, such as up to 30 centimeters away from the closest healthy nerve.

Compositions of the disclosure may be suspended in a hydrogel solution, e.g., for topical application. Examples of suitable hydrogels include self-assembling peptides, such as RAD16. Alternatively, the hydrogel solution containing the therapeutic agent may be allowed to harden to form a matrix prior to application. The hydrogel may be an organic polymer (natural or synthetic) that is cross-linked via covalent, ionic, or hydrogen bonds to create a three-dimensional open-lattice structure that entraps water molecules to form a gel. Examples of materials that can be used to form a hydrogel include collagen, hyaluronate, polysaccharides such as alginate and salts thereof, peptides, polyphosphazines, and polyacrylates, which are crosslinked ionically, block polymers such as polyethylene oxide-polypropylene glycol block copolymers which are crosslinked by temperature or pH, respectively, or coacervates.

In a particular embodiment, the composition is topically applied to the wound, for example a diabetic foot ulcer, as a lyophilized, hygroscopic powder. The wound may then be covered with appropriate wound dressings.

Compositions of the disclosure may also be included in dressings or scaffolds to facilitate application to wounds. The compositions may also be aqueous solutions that are sprayed on the wound.

In particular embodiments, compositions of the disclosure are used to treat or prevent nerve injury, such as neuropathy. The wound may be due to any number of causes including but not limited to a surgical wound, skin lesion, a burn, an injury, a graft wound, or a diabetic wound. The skin lesion may be a venous ulcer, diabetic ulcer, pressure sore, burn or iatrogenic grafting wound. Neuropathies or other nerve injuries that can benefit from the healing activity of the therapeutic agents of the disclosure include, without limitation, those resulting from, for example, diabetes, ischemic events, lacerations, crush injuries, HIV, surgical intervention, radiation or chemotherapy.

In particular embodiments, compositions of the disclosure are further used to promote or stimulate angiogenesis or revascularization, e.g., at a site of injury or tissue damage.

The compositions of the disclosure may be used alone or in combination with one or more other therapeutic agents or procedures to treat or prevent an injury or disease. For example, in certain embodiments, to enrich blood supply to a damaged tissue and/or to promote tissue regeneration, compositions of the disclosure may be used in combination with platelet-rich plasma. When used in combination with one or more other therapeutic agents or procedures, the compositions of the disclosure may be provided or used prior to, at the same or during an overlapping time period as, or subsequent to, treatment with the other therapeutic agent or procedure.

When used in combination with another therapeutic agent, a composition of the disclosure may be provided separately from the other agent, or it may be present in a pharmaceutical composition that also contains the other therapeutic agent, e.g., a co-formulation comprising two or more therapeutic agents, one being the composition of the disclosure. In particular embodiments, the composition of the disclosure and an additional therapeutic agent are both combined with or associated with the same implant, matrix or scaffold.

The compositions of the invention are administered in a therapeutically effective amount, which will vary depending upon a variety of factors including but not limited to the activity of the specific composition employed; the age, body weight, general health, sex and diet of the subject to which the composition of the invention is administered; the mode and time of administration; the rate of excretion, effusion, or breakdown of the composition in the subject; and the type, size, or severity of the injury, disease, or condition to be treated. The compositions of the disclosure comprise, in some embodiments, higher concentration of particular factors than found in otherwise comparable MCP or processed microvascular tissue compositions. For example, the compositions deliver greater than 10⁵ CD90+ cells per cm² of the wound (or the contents of such CD90+ cells when the cells are further processed before administration rupturing cell membranes). In certain embodiments, the compositions deliver over 1000 fibroblast colony-forming units (CFU-F) per cm² (or contents of a cellular composition having 1000 CFU-F but further processed). In yet other embodiments, the compositions permit delivery of >10 pg of bFGF, BDNF, and/or HDGF with <10 pg TGF-01 per cm² of the wound.

The methods of the disclosure may be practiced by administering a therapeutic agent or a composition comprising one or more therapeutic agents in one, two or more doses. For example, in certain embodiments, a therapeutic agent or a composition comprising one or more therapeutic agents is administered as a single dose, multiple doses or in repeated doses over a period of time. The therapeutic agent or the composition comprising one or more therapeutic agents may be administered every 3 hours, every day, every 3 days, every 7 days, every 10 days, every 14 days, every 28 days or even longer intervals as the patient requires.

The methods summarized above and set forth in further detail below describe certain actions taken by a practitioner; however, it should be understood that they can also include the instruction of those actions by another party. Thus, actions such as “administering microvascular tissue” include “instructing the administration of microvascular tissue.”

### EXAMPLES

### Example 1: Characterization of a Multipotent Cell Preparation

The aim of these experiments was to develop and test convenient new products for treating DFU based upon multipotent cells. We targeted a dose of at least 1000 CFU-F or 10⁵ CD90⁺ cells per square cm. We used multipotent cells isolated from human adipose tissue after mincing the fat, digesting with collagenase and neutral protease, and lysing red blood cells with ammonium chloride.

A batch of multipotent cells was produced from human adipose tissue and vialed and then characterized for phenotype and some growth factors of interest at contract labs. Cell count and phenotype were measured by INCELL, Inc. (San Antonio, Tex.) while growth factors were measured by ELISA at AssayGate (Ijamsville, Md.). The phenotype results show 5.5×10⁵ CD90⁺ cells/vial.

To remove red blood cells, the sample was subjected to ammonium chloride solution. The preparation was assayed by ELISA in a total volume of 1 ml and gave the results tabulated below. The HDGF (hepatoma derived growth factor) was quantified at the Proteomics Lab at Colorado State Univ.

TABLE 3

| Growth Factor | Quantity/vial | |
|---|---|---|
| bFGF | 558 | pg |
| BDNF | 40 | pg |
| TGF-β₁ | <6.5 | pg |
| HDGF | 24 | ng |

### Example 2: Topical Delivery to a Wound in a Limb with Neuropathy

The preparation of multipotent cells as characterized in Example 1 was lyophilized and sterilized by gamma irradiation. The resulting cake is porous, brittle and easily crushed to powder. The powder was sprinkled (topically) on diabetic foot ulcers after debridement and cleaning per standard wound care practice. The wounds were then covered with an occlusive dressing (Adaptic Touch™, San Antonio, Tex.) held in place with Steri-strips (3M, St. Paul, Minn.). Two patients received the multipotent cell preparation while a control patient received the same care without the addition of the multipotent cells. Healing of the DFU was measured weekly before reapplication of the multipotent cells and dressings. The extent of the patients' peripheral neuropathy was measured using the Von Frey method, where a monofilament fiber that bends at a calibrated force is used to probe the limb, moving up the leg until the patient can feel the fiber.

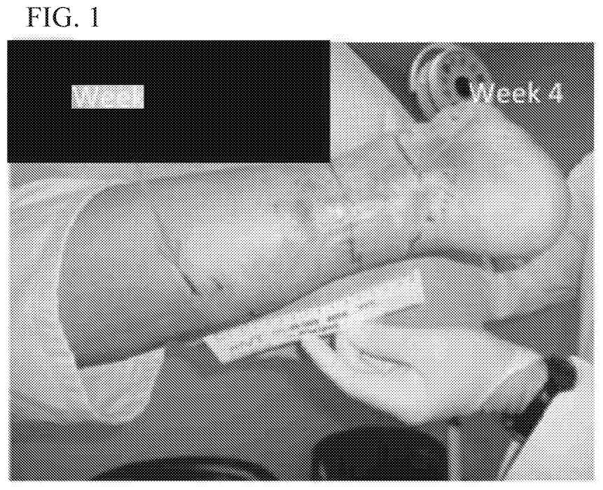

FIG. 1 shows results for Patient A who presented with a diabetic foot ulcer on the bottom of his foot that had not healed despite standard care with hydrocolloid dressings. After debridement, the ulcer measured 5.1 cm² and the neuropathy extended halfway to the knee. Four weeks later, the DFU measured 3.5 cm² and the extent of the neuropathy was reduced by about 14 cm. In this patient the reversal of neuropathy was accompanied by rather severe pain requiring medication. Weekly application of the therapeutic agent was stopped when the DFU fully healed, but the neuropathy continued to heal and the associated pain fully resolved subsequently.

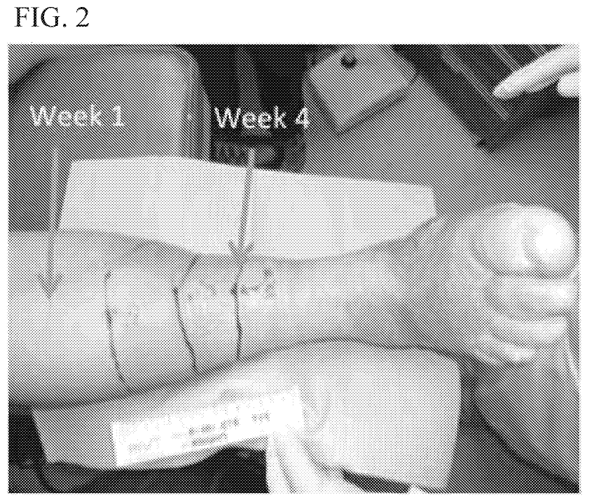

FIG. 2 shows results for Patient B who presented with a diabetic foot ulcer on the medial side of her foot that had not healed despite standard care with hydrocolloid dressings. After debridement the ulcer measured 6.1 cm² and the neuropathy extended over halfway to the knee. Four weeks later the DFU measured 0.4 cm² and the extent of the neuropathy was reduced by about 16 cm. Her wound did fully heal and the improvement in neuropathy persisted after completion of the treatments with the multipotent cell preparation.

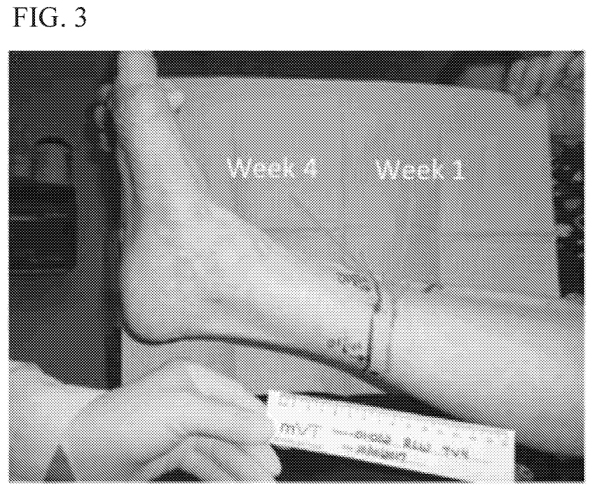

FIG. 3 shows control data in which Patient C presented with a diabetic foot ulcer on the bottom of her foot that had not healed despite standard care with hydrocolloid dressings. After debridement the ulcer measured 4.0 cm² and the neuropathy extended ⅓ of the distance to the knee. Four weeks later, the DFU measured 1.6 cm² and the extent of the neuropathy was reduced by about 1 cm. At 12 weeks, the DFU had not healed and there was little change in her neuropathy.

As illustrated in FIGS. 1 and 2, the multipotent cell treatments had a dramatic impact on the patients' neuropathy. By week 5, both patients A and B had some feeling on the bottom of their feet for the first time in years. In contrast, as shown in FIG. 3, control patient C saw little effect.

### Example 3: Subcutaneous Injection in Limb with Neuropathy

An adult male with diabetic neuropathy had lost much of his feeling below his left ankle. He was treated during one visit with 4 subcutaneous injections of the same preparation that was used in Example 2. Each injection consisted of 275,000 CD90⁺ cells dissolved in 1 ml of normal saline. The patient later reported a 60% improvement in sensation in his left foot and the improvement extended from his ankle to the region of the injections. Thus, the preparation did have a beneficial impact on the neuropathy, but it was not as pronounced as when the agent was applied topically to a DFU in the previously described patients.

1. A method of treating, preventing, improving, or reducing peripheral neuropathy in a subject in need thereof, comprising
administering to a subject a composition comprising a therapeutic agent,
wherein the subject has at least one damaged nerve in an affected region
wherein the administering step comprises topically administering the composition to the affected region.

2. The method of claim 1, wherein the therapeutic agent comprises, per milliliter, one or more cells or proteins selected from:
a) at least about 1000 CFU-F cells,
b) the contents of at least about 1000 CFU-F cells,
c) at least about 10⁵ CD90 positive cells,
d) the contents of at least about 10⁵ CD90 positive cells,
e) at least about 10 picograms (pg) HDGF,
f) at least about 10 pg bFGF,
g) at least about 10 pg BDNF,
h) at least about 10 pg of bFGF and BDNF, collectively, and
i) at most about 10 pg TGF-b₁.

3. The method of claim 1 or claim 2, wherein the subject suffers from diabetes or the peripheral neuropathy comprises diabetic neuropathy.

4. The method of any one of claims 1 to 3, wherein the subject has one or more diabetic foot ulcers (DFU).

5. The method of any one of claims 1 to 4, wherein the administering step comprises topically administering the composition to the subject at least twice in three months.

6. The method of any one of claims 1 to 5, wherein the administering step comprises topically administering the composition to the subject every 3, 4, 5, 6, 7, 8, 9, 10, 11, 12, 13, 14, 15, 16, 17, 18, 19, or 20 days.

7. The method of any one of claims 1 to 6, wherein the affected region comprises a wound, optionally an ulcer.

8. The method of any one of claims 1 to 7, wherein the therapeutic agent comprises a multipotent cell preparation (MCP) selected from the group consisting of processed microvascular tissue, a mesenchymal stem cell (MSC) preparation, stromal vascular fraction (SVF) from adipose tissue, a bone marrow cell preparation, and umbilical cord stem cells.

9. The method of any one of claims 1 to 7, wherein the therapeutic agent comprises processed microvascular tissue, optionally mVASC®.

10. The method of any one of claims 1 to 9, wherein the processed microvascular tissue is derived from human adipose tissue.

11. The method of any one of claims 1 to 10, wherein the processed microvascular tissue is processed with one or more treatment selected from:
a) collagenase treatment,
b) neutral protease treatment,
c) cell lysis, optionally ammonium chloride cell lysis,
d) drying, optionally freeze-drying, air drying, or lyophilization,
e) freezing, optionally freeze-drying or flash freezing, and
f) irradiation, optionally gamma irradiation or electron-beam irradiation.

12. The method of any one of claims 1 to 11, wherein the composition is formulated for topical administration.

13. The method of any one of claims 1 to 12, wherein the composition comprises a powder, a crushable cake, a film, a gel, an ointment, a suspension, an emulsion, a concentrate, a coacervate, a scaffold, a hygroscopic powder, or a wound dressing or covering.

14. The method of any one of claims 1 to 13, wherein administering the composition to the subject results in adherence of the composition to the affected region.

15. The method of any one of claims 1 to 14, wherein the method results in one or more of improved nerve sensation, tissue healing, wound closure, ulcer healing, prevention of wound recurrence, increased flexibility, or decreased pain.

16. The method of any one of claims 1 to 15, wherein the method improves healing of the ulcer, improves nerve sensation in the region, or reduces the recurrence rate of new ulcers in the region.

17. The method of any one of claims 1 to 16, wherein the affected region is within 5, 10, 15, 30 cm of a damaged nerve.

18. A composition for treating, preventing, improving, or reducing peripheral neuropathy in a subject in need thereof, comprising a therapeutic agent, wherein the therapeutic agent comprises, per milliliter, one or more cells or proteins selected from:
a) at least about 1000 CFU-F cells,
b) the contents of at least about 1000 CFU-F cells,
c) at least about 10⁵ CD90 positive cells,
d) the contents of at least about 10⁵ CD90 positive cells,
e) at least about 10 picograms (pg) HDGF,
f) at least about 10 pg bFGF,
g) at least about 10 pg BDNF,
h) at least about 10 pg of bFGF and BDNF, collectively, and
i) at most about 10 pg TGF-b₁.

19. The composition of claim 18, wherein the subject suffers from diabetes or the peripheral neuropathy comprises diabetic neuropathy.

20. The composition of claim 18 or claim 19, wherein the subject has one or more diabetic foot ulcers (DFU).

21. The composition of any one of claims 18 to 20, wherein the administering step comprises topically administering the composition to the subject at least twice in three months.

22. The composition of any one of claims 18 to 21, wherein the administering step comprises topically administering the composition to the subject every 3, 4, 5, 6, 7, 8, 9, 10, 11, 12, 13, 14, 15, 16, 17, 18, 19, or 20 days.

23. The composition of any one of claims 18 to 22, wherein the affected region comprises a wound, optionally an ulcer.

24. The composition of any one of claims 18 to 23, wherein the therapeutic agent comprises a multipotent cell preparation (MCP) selected from the group consisting of processed microvascular tissue, a mesenchymal stem cell (MSC) preparation, stromal vascular fraction (SVF) from adipose tissue, a bone marrow cell preparation, and umbilical cord stem cells.

25. The composition of any one of claims 18 to 23, wherein the therapeutic agent comprises processed microvascular tissue, optionally mVASC®.

26. The composition of any one of claims 18 to 25, wherein the processed microvascular tissue is derived from human adipose tissue.

27. The composition of any one of claims 18 to 26, wherein the processed microvascular tissue is processed with one or more treatment selected from:
g) collagenase treatment,
h) neutral protease treatment,
i) cell lysis, optionally ammonium chloride cell lysis,
j) drying, optionally freeze-drying, air drying, or lyophilization,
k) freezing, optionally freeze-drying or flash freezing, and
l) irradiation, optionally gamma irradiation or electron-beam irradiation.

28. The composition of any one of claims 18 to 27, wherein the composition is formulated for topical administration.

29. The composition of any one of claims 18 to 28, wherein the composition comprises a powder, a crushable cake, a film, a gel, an ointment, a suspension, an emulsion, a concentrate, a coacervate, a scaffold, a hygroscopic powder, or a wound dressing or covering.

30. The composition of any one of claims 18 to 29, wherein administering the composition to the subject results in adherence of the composition to the affected region.

31. The composition of any one of claims 18 to 30, wherein the composition results in one or more of improved nerve sensation, tissue healing, wound closure, ulcer healing, prevention of wound recurrence, increased flexibility, or decreased pain.

32. The composition of any one of claims 18 to 31, wherein the composition improves healing of the ulcer, improves nerve sensation in the region, or reduces the recurrence rate of new ulcers in the region.

33. A method of treating, preventing, improving, or reducing peripheral neuropathy in a subject in need thereof, comprising
administering to subject a composition comprising a therapeutic agent,
wherein the subject has at least one wound in an affected region, and
wherein the administering step comprises topically administering the composition to the wound in the affected region.

34. The method of claim 33, wherein the topical administration provides, per cm² of wound, one or more cells or proteins selected from:
j) at least about 1000 CFU-F cells,
k) the contents of at least about 1000 CFU-F cells,
l) at least about 10⁵ CD90 positive cells,
m) the contents of at least about 10⁵ CD90 positive cells,
n) at least about 10 picograms (pg) HDGF,
o) at least about 10 pg bFGF,
p) at least about 10 pg BDNF,
q) at least about 10 pg of bFGF and BDNF, collectively, and
r) at most about 10 pg TGF-b₁.

35. The method of claim 33 or claim 34, wherein the subject suffers from nerve injury, damaged nerves, diabetes, or diabetic neuropathy.

36. The method of any one of claims 33 to 35, wherein the at least one wound comprises a diabetic foot ulcer (DFU).

37. The method of any one of claims 33 to 36, wherein the administering step comprises topically administering the composition to the subject at least twice in three months.

38. The method of any one of claims 33 to 37, wherein the administering step comprises topically administering the composition to the subject every 3, 4, 5, 6, 7, 8, 9, 10, 11, 12, 13, 14, 15, 16, 17, 18, 19, or 20 days.

39. The method of any one of claims 33 to 38, wherein the wound comprises an ulcer.

40. The method of any one of claims 33 to 39, wherein the therapeutic agent comprises a multipotent cell preparation (MCP) selected from the group consisting of processed microvascular tissue, a mesenchymal stem cell (MSC) preparation, stromal vascular fraction (SVF) from adipose tissue, a bone marrow cell preparation, and umbilical cord stem cells.

41. The method of any one of claims 33 to 39, wherein the therapeutic agent is selected from the group consisting of nerve growth factor (NGF), glial cell line derived neurotrophic factor (GDNF), J147, curcumin, and Insulin-like growth factor 1 (IGF-1) or a derivative thereof.

42. The method of any one of claims 33 to 39, wherein the therapeutic agent comprises processed microvascular tissue, optionally mVASC®.

43. The method of claim 42, wherein the processed microvascular tissue is derived from human adipose tissue.

44. The method of claim 42 or claim 43, wherein the processed microvascular tissue is processed with one or more treatment selected from:
m) collagenase treatment,
n) neutral protease treatment,
o) cell lysis, optionally ammonium chloride cell lysis,
p) drying, optionally freeze-drying, air drying, or lyophilization,
q) freezing, optionally freeze-drying or flash freezing, and
r) irradiation, optionally gamma irradiation or electron-beam irradiation.

45. The method of any one of claims 33 to 44, wherein the composition is formulated for topical administration.

46. The method of any one of claims 33 to 45, wherein the composition comprises a powder, a crushable cake, a film, a gel, an ointment, a suspension, an emulsion, a concentrate, a coacervate, a scaffold, a hygroscopic powder, or a wound dressing or covering.

47. The method of any one of claims 33 to 46, wherein administering the composition to the subject results in adherence of the composition to the affected region.

48. The method of any one of claims 33 to 47, wherein the method results in one or more of improved nerve sensation, tissue healing, wound closure, ulcer healing, prevention of wound recurrence, increased flexibility, or decreased pain.

49. The method of any one of claims 33 to 48, wherein the method improves healing of the ulcer, improves nerve sensation in the region, or reduces the recurrence rate of new ulcers in the region.
